(19) United States
(12) Patent Application Publication
(10) Pub. No.: US 2002/0111556 A1
(43) Pub. Date: Aug. 15, 2002

(54) Method for recording electrocardiograms

(76) Inventor: Stanley Wegner, Auburn, CA (US)

Correspondence Address: STANLEY WEGNER, 26424 TABLE MEADOW RD., AUBURN, CA, 95602, US

(21) Appl. No.: 10/075,527

(22) Filed: Feb. 12, 2002

Related U.S. Application Data
(60) Provisional application No. 60/268,531, filed on Feb. 15, 2001.

(51) Int. Cl. A61B 5/04

(52) U.S. Cl. 600/523

(57) ABSTRACT

A new method for storing electrocardiogram (ECG) data in Transtelephonic/Cardiac Event Recorders is described. This method converts the ECG to a frequency-modulated (FM) format within the audible range and stores the FM converted ECG data in an audio-data recording device for subsequent acoustic FM telephone transmission to an ECG receiving center. The new method does not require analog-digital or digital-analog converters as used by conventional ECG recording devices and offers improved ECG resolution and dynamic range.

### CROSS-REFERENCE TO RELATED APPLICATIONS

This application claims priority from the applicant's co-pending Provisional Application Serial No. 60/268,531, filed Feb. 15, 2001.

### BACKGROUND OF THE INVENTION

1. Technical Field

The present invention relates to the field of medical instrumentation, and more particularly, to a branch of electrocardiography referred to as Transtelephonic Electrocardiogram (ECG) recording and/or Cardiac Event Recording. This technology utilizes small, hand-held devices to record and store an ECG wave-form in a digital memory for subsequent telephone-transmission to a receiving center for analysis by medical personnel. These devices are prescribed by physicians for evaluating possible cardiac-related symptoms. The method described herein relates to a unique method of converting and storing the ECG signal for use in transtelephonic/cardiac event recorders.

2. Background

Transmitting ECG by telephone was first published by Wilhelm Einthoven in Archives Internationale de Physiologie, 4:132, 1906. A translation of this work was published in the U.S. literature by Dr. Henry Blackburn (Blackburn, Henry W.: Translation of “Le Telecardiogram ”, American Heart Journal, 54:602,) in April, 1957. In the ensuing years, numerous instruments were developed to record and transmit ECGs from patient's homes, hospitals, and physician's offices. Examples of such instruments are described in U.S. Pat. No. 3,199,508 filed in 1965 and U.S. Pat. No. 3,991,747 filed in 1974. These devices had no storage capability and could only monitor “real-time” ECGs. Their primary application was monitoring cardiac pacemaker operation remotely from patients' homes.

The development of low-power solid-state memories (Random Access Memories RAMs) in the 1980's gave rise to the current recorder technology, which stores the ECG signal in digital form in memory. Recordings can thus to be made whenever required, e.g., when a patient experiences symptoms, and transmitted at a later time to the analysis center. This technology, referred to as Transtelephonic ECG Monitoring or Cardiac Event Recording, has now become a standard diagnostic technique for evaluating ECGs in patients with infrequent symptoms. The patient can record his/her ECG whenever the symptom occurs using a pager-sized recorder and later transmit the stored ECG to an analysis center for evaluation.

3. Prior Art

Although transmission of ECGs by telephone is an old technology, first published by the founder of modern electrocardiography—Wilhelm Einthoven—in 1906 (Archives Internationale de Physiologie, 4:132), the practical application of this technology awaited the development of battery-powered storage devices. These devices enabled patients to record ECGs during symptomatic events no matter when or where they occurred. Commercial offerings of devices incorporating solid-state memory were made in the early 1980's. U.S. Pat. Nos. 4,483,346 and 4,531,527 describe ECG recording and transmission systems incorporating digital memory. Since that time, numerous American and International manufacturer's have produced memory cardiac event recorders.

Recent developments in this field have focused on the physical design of the devices—e.g., monitors incorporated into wrist-watches as revealed in U.S. Pat. Nos. 5,191,891, 5,333,616 and 5,365,935). The common features of these and previous devices are: 1) the use of analog-digital conversion of the ECG, 2) storage of the digitized data in a solid-state memory, such as RAM, EPROM, or FLASH Memory, and 3) digital to analog conversion and frequency modulation (FM) for playback. No devices have been reported which store ECG in an FM format, the method described herein, which was previously disclosed in Provisional U.S. Patent Application No. 60/268,531, on Feb. 15, 2001.

### BRIEF SUMMARY OF THE INVENTION

The unique method disclosed herein for storage of ECG data in transtelephonic devices makes use of a new technology developed for storing voice recordings and used in telephone answering systems, voice recorders, etc. as illustrated in U.S. Pat. Nos. 4,890,259, 5,241,494 and 5,294,819. This technology, termed “digital-analog recording”, employs a high-density EEPROM in which each cell has 256 levels instead of the usual binary (on/off state) information. The device, called a ChipCorder® (ChipCorder® is a registered trademark of Information Storage Devices, Inc.), can store and reproduce sound with high precision. U.S. Pat. No. 4,890,259 describes the advantages of this technology over conventional analog-digital conversion techniques in relation to audio recording applications.

To use this storage device, the method described here first converts the ECG to a constant-amplitude Frequency-Modulated (FM) format within the audible frequency range (e.g., 1800 Hz) and stores the FM data as “sound” in the ChipCorder®. The present invention eliminates the need for A/D and D/A converters used in conventional systems and improves the resolution and input signal range of conventional recording techniques. Using the input voltage range of 4 millivolts (+/−2 mV) recommended by the Association for the Advancement of Medical Instrumentation, 3330 Washington Blvd., Arlington, Va. 22201, (American National Standard-Ambulatory Electrocardiographs, ANSI/AAMI EC38-1994), the quantization error of a typical 8-Bit digital system would be 31.25 microvolts (i.e., +/−1 bit) whereas the ChipCorder® gives a stability of better than 5 microvolts. The commonly used modulation level of 100 Hz/millivolt for conventional recording systems allows an input voltage range for the present invention in excess of 10 millivolts (i.e., +/−5 millivolts) as the modulated frequencies are well within the bandwidth of standard telephone lines (1800 Hz +/−500 Hz=1300 Hz−2300 Hz). Alternatively, modulation could be doubled (200 Hz/millivolt) with the accepted input voltage range to improve resolution with conventional receiving systems. Moreover, the non-volatile memory permits retrieval of recorded data in event of battery failure.

### BRIEF DESCRIPTION OF THE SEVERAL VIEWS OF THE DRAWING

FIG. 1 is a diagram showing the conventional, prior art, method of recording and storing ECG data.

FIG. 2 is a diagram of the method of recording and storing ECG data as revealed in the present invention.

### DETAILED DESCRIPTION OF THE INVENTION

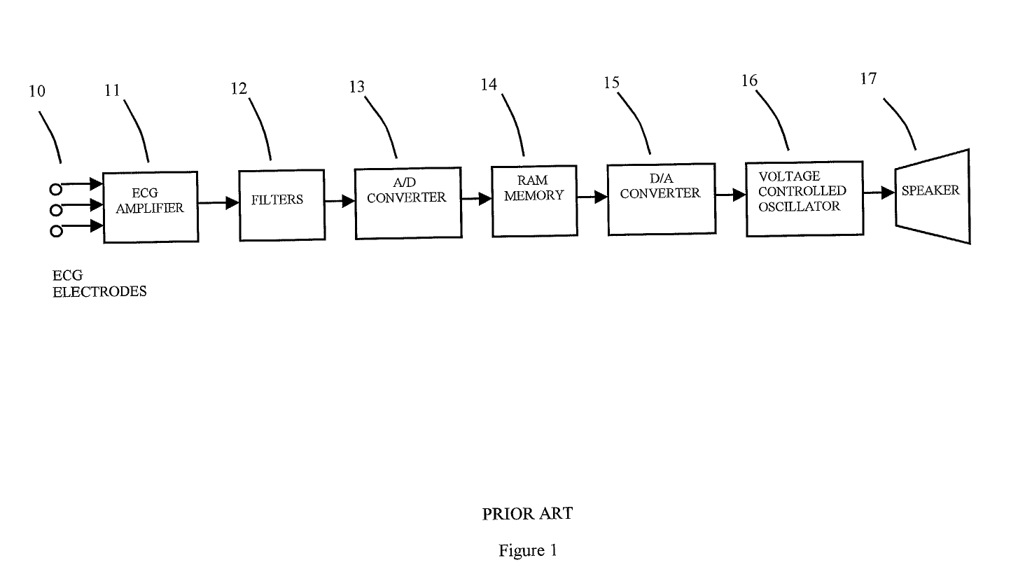

In a conventional prior art system, as shown in FIG. 1, the ECG signal is sensed from the body surface by electrodes 10, amplified 11, band-pass filtered 12, digitized 13, and stored in digital memory 14 as an 8-bit value (10 and 12-bit systems have also been used). On playback, the stored digital wave-form is re-converted to analog form 15, converted to a frequency-modulated signal 16, and played through a speaker 17 for acoustic transmission over telephone lines to a receiving system.

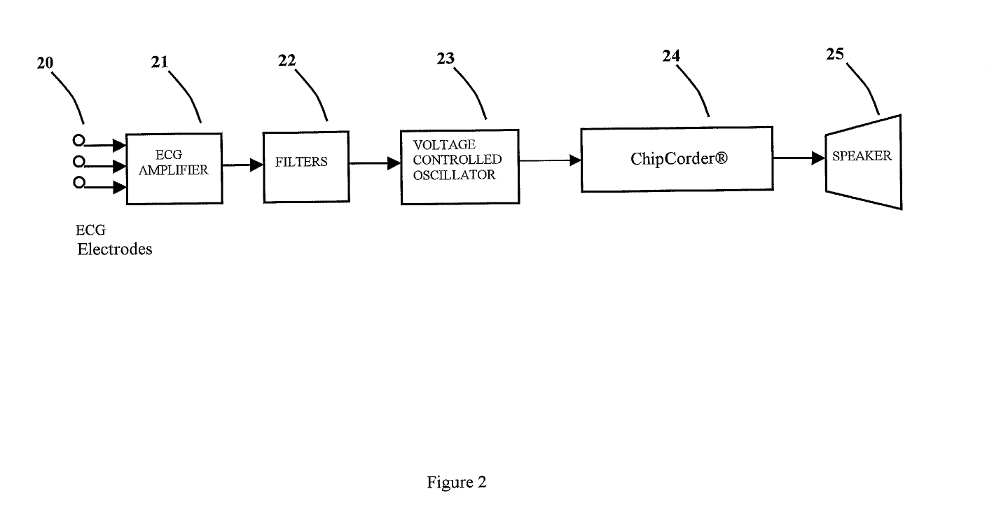

The method of the present invention for ECG recording and storage, is shown in FIG. 2. Referring to FIG. 2, the ECG signal is sensed from the body surface by electrodes 20, is amplified 21, band-pass filtered 22, but in contrast to the conventional method, is converted to a constant-amplitude Frequency-Modulated signal within the audible range (e.g., 1800 Hz) by the voltage-controlled oscillator 23. The FM signal is then stored as a “sound” in the ChipCorder® 24. On playback, the recorded FM signal is output is applied to the speaker 25 for acoustic telephone-transmission of the ECG data to a receiving system. The ChipCorder® in FIG. 2 replaces the A/D converter 13, RAM memory 14, and the D/A converter 15 in a conventional system (FIG. 1).

I claim:

1. A system for storage of electrocardiogram (ECG) data comprised of an ECG monitoring device adapted to record patient-generated ECG data, convert said data to Frequency-modulated format, electronically store said data in a voice-storage system, subsequently recall said data, and transmit said data acoustically by telephone to a receiving station for demodulation to the original ECG wave-form.

2. The system of claim 1 wherein ECG data is converted to a constant-amplitude FM signal within the audible frequency range and stored as acoustic data in a non-volatile audio-data storage device. In the preferred embodiment of the invention, a ChipCorder® is used as the ECG-data storage device.

3. The system of claim 1 wherein ECG data is stored in non-volatile memory without the requirement of an A/D converter.

4. The system of claim 1 wherein ECG data is retrieved from the non-volatile audio-data storage device in FM format and transmitted acoustically by telephone to a receiving station.

5. The system of claim 1 wherein ECG data is stored is retrieved from non-volatile memory and applied to a speaker for acoustic ECG transmission without the requirement of a D/A converter.
